(19) United States
(12) Patent Application Publication
(10) Pub. No.: US 2018/0126022 A1
(43) Pub. Date: May 10, 2018

(54) TABLE-TOP HEAT-ACTIVATED AROMA DIFFUSER

(71) Applicant: DONGGUAN YIH TEH ELECTRIC PRODUCTS CO., LTD., Dongguang (CN)

(72) Inventor: Ming Jen HSIAO, Miaoli County (TW)

(21) Appl. No.: 15/862,356

(22) Filed: Jan. 4, 2018

Related U.S. Application Data
(63) Continuation-in-part of application No. 14/938,564, filed on Nov. 11, 2015.
Application No. 14/938,564 is a continuation-in-part of application No. 15/258,757, filed on Sep. 7, 2016.
Application No. 15/258,757 is a continuation-in-part of application No. 15/653,362, filed on Jul. 18, 2017.

(51) Int. Cl. A61L 9/03; A61L 9/04; H05B 3/26; F21V 15/01; F21V 33/00

(52) U.S. Cl. CPC A61L 9/03; A61L 9/042; H05B 3/26; F21Y 2115/10; F21V 33/0028; H05B 2203/021; F21V 15/01

(57) ABSTRACT

A table-top heat-activated aroma diffuser includes a light-transmissive housing including a first opening located on one side thereof and a second opening located on an opposite side thereof, a heat-transfer unit mounted in the first opening of the light-transmissive housing, a heater mounted to a bottom side of the heat-transfer unit for heating an aroma capsule being placed on the heat-transfer unit to release an aromatic vapor, and a light-emitting device mounted inside the light-transmissive housing for creating halo light soft decorative effect.

### CROSS-REFERENCES TO RELATED APPLICATION

The present invention is a continuation-in-part of patent application Ser. No. 14/938,564 filed on Nov. 11, 2015 and Ser. No. 15/258,757 filed on Sep. 7, 2016 and patent application Ser. No. 15/653,362 filed on Jul. 18, 2017.

### BACKGROUND OF THE INVENTION

### 1. Field of the Invention

The present invention relates to scent releasing devices and more specifically, to table-top heat-activated aroma diffuser using a replaceable multilayer breathing film-based aroma capsule.

### 2. Description of the Related Art

U.S. Pat. No. 8,066,420, U.S. Pat. No. 8,262,277, U.S. Pat. No. 8,147,116 disclose similar aroma diffusing night lamp system designs. However, the light-emitting means in these designs cannot emit light in all directions. Further, these designs do not provide light guide means to create a good atmosphere of halo light soft decorative effect. Further, the arrangement of the power supply, heat source and essential oil or fluid aromatic substance is not safety. In the use of aforesaid prior art designs, it is inconvenient to refurnish the aromatic substance. When in use, the user needs to take the aromatic substance out of the heat-transfer container carefully, and then to put the aromatic substance in the top accommodation open chamber of the table-top heat-activated aroma diffuser, avoiding direct contact of the hands with the aromatic substance. Further, after the aromatic substance is used up, the user needs to clean the heat-transfer container. If the heat-transfer container is not well cleaned after each use, a new supply of aromatic substance will be mixed with the residual aromatic substance in the accommodation chamber, giving off a bad smell. Further, if the heat-transfer container is made of a fragile material (for example, ceramics or glass), it may be forced down or broken easily during cleaning, causing the aromatic substance to fall to the ground or to the fluid aromatic substance to contaminate the table-top heat-activated aroma diffuser or the surroundings.

Therefore, it is desirable to provide an aroma-diffusing design that eliminates the drawbacks of the aforesaid prior art designs.

### SUMMARY OF THE INVENTION

The present invention has been accomplished under the circumstances in view. It is therefore an object of the present invention to provide a table-top heat-activated aroma diffuser, which can simply and smoothly combine a disposable aroma capsule that can be discarded after use and that allows replacement in a rapid manner.

To achieve this and other objects of the present invention, a table-top heat-activated aroma diffuser comprises a light-transmissive housing, a heat-transfer unit and a heater. The heat-transfer unit is mounted in a first opening in one side of the light-transmissive housing. The heater is mounted to a bottom side of the heat-transfer unit and electrically connected to a power supply unit for heating the heat-transfer unit.

When using the heat-activated aroma diffuser, the user can place an aroma capsule on the heat-transfer unit. The power supply unit provides electricity to the heater for causing the heater to generate heat. The heat energy generated by the heater is then transferred by the heat-transfer unit to the aroma capsule, causing the aroma capsule to release an aromatic vapor.

Preferably, the table-top heat-activated aroma diffuser further comprises a light guide and a light-emitting device. The light guide is mounted inside the light-transmissive housing. The light-emitting device is mounted in the light guide and electrically connected to a power source. When the light-emitting device is activated to emit light, the light guide guides the emitted light to the light-transmissive housing toward the outside of the table-top heat-activated aroma diffuser.

Preferably, the heat-activated aroma diffuser further comprises a connection unit and a base. The base is fastened to an opposite side of the light-transmissive housing. The heater has one side thereof attached to a bottom side of the heat-transfer unit. The connection unit has one end thereof fastened to the base, and an opposite end thereof stopped against an opposite side of the heater. The heater is mounted in a bracket at the bottom side of the heat-transfer unit.

The heater can be a positive temperature coefficient (TPC) thermistor, ceramic resistor, or any other heating resistor.

Preferably, the table-top heat-activated aroma diffuser combines an aroma capsule. The aroma capsule is placed on the heat-transfer unit inside the light-transmissive housing. Further, the aroma capsule comprises a disposable heat-transfer container, an aromatic substance contained in the disposable heat-transfer container.

The aromatic substance is selected from the group consisting of aromatic wax, perfume, balsam, sesame oil mixture and essential oil that release an aromatic vapor when heated.

When using the table-top heat-activated aroma diffuser, the user places the aroma capsule on the heat-transfer unit, and the aroma capsule will release a pleasant aromatic vapor when heated.

After the aromatic substance is used up, the heat-transfer container can be thrown away or recycled, and a new aroma capsule can be put in the table-top heat-activated aroma diffuser. When the light-emitting device is activated to emit light, the emitted light is transmitted through the light guide to create a halo light soft decorative effect. At the same time, the light-transmissive housing emits an aromatic vapor out of the heat-activated aroma diffuser to create a good atmosphere. Thus, the heat-activated aroma diffuser works as a night lamp, reminding the user that the heat-activated aroma diffuser is in use.

### BRIEF DESCRIPTION OF THE DRAWINGS

FIG. 1 is a sectional side view of a table-top heat-activated aroma diffuser in accordance with the present invention.

FIG. 2 is an exploded view of the table-top heat-activated aroma diffuser in accordance with the present invention.

FIG. 3 is an oblique top elevational view of the table-top heat-activated aroma diffuser in accordance with the present invention.

FIG. 4 is a side view of the table-top heat-activated aroma diffuser in accordance with the present invention.

### DETAILED DESCRIPTION OF THE PREFERRED EMBODIMENT

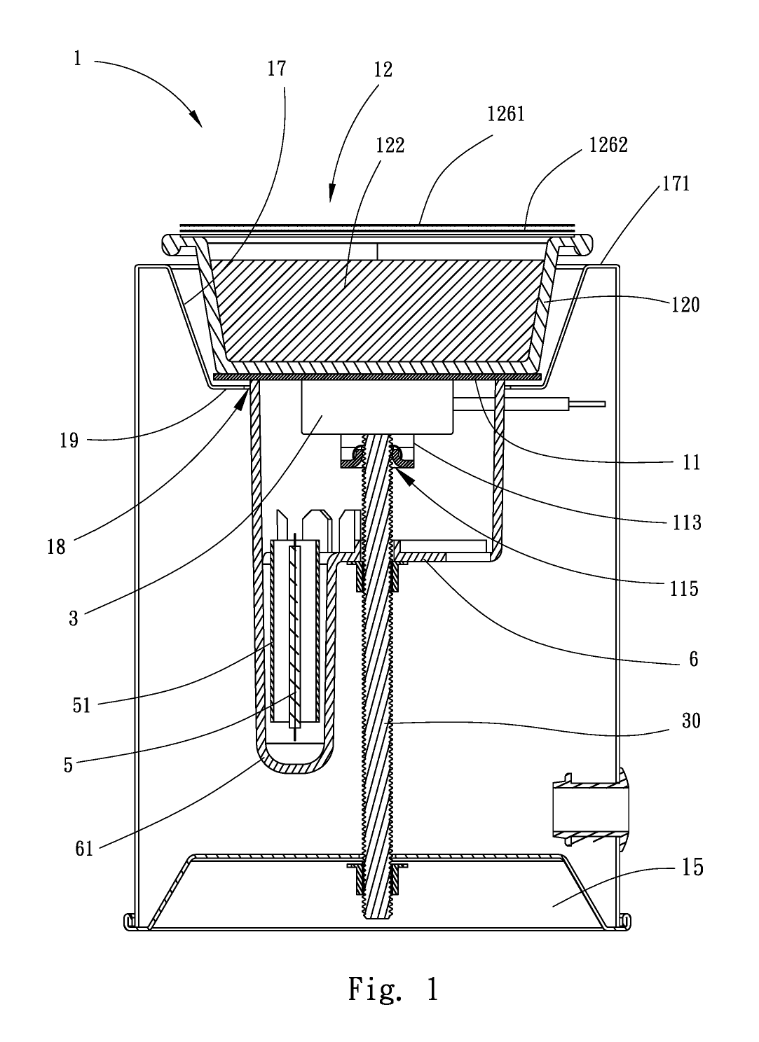

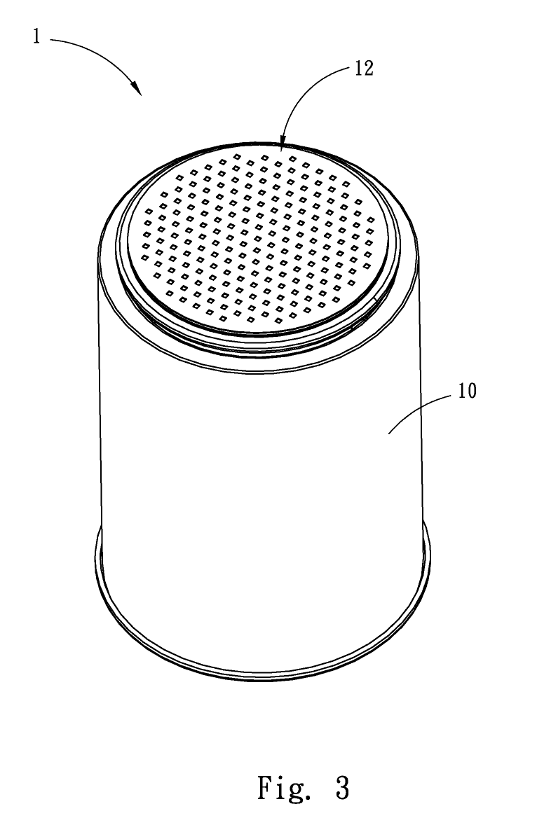

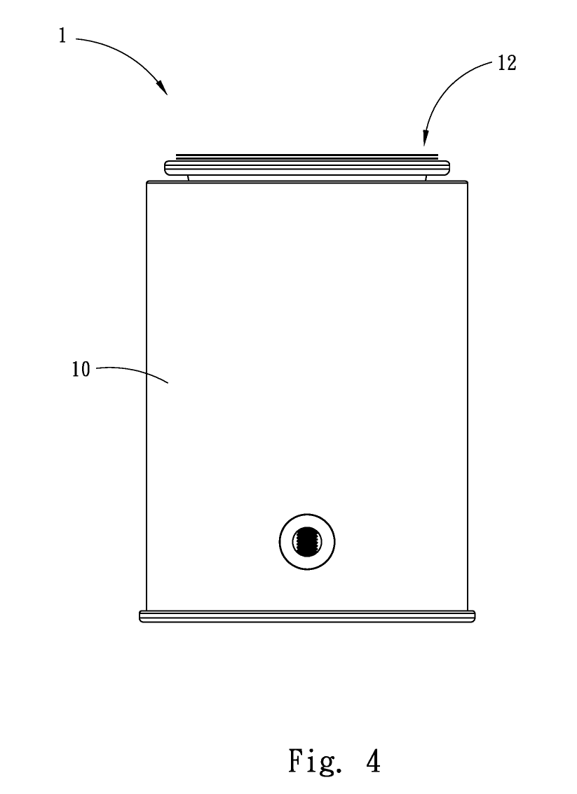

Referring to FIGS. 1-6, a table-top heat-activated aroma diffuser 1 in accordance with the present invention is shown. The table-top heat-activated aroma diffuser 1 comprises a light-transmissive housing 10, a heat-transfer unit 11, a heater 3, and a light-emitting device 5. The light-transmissive housing 10 in the present preferred embodiment is a hollow shell, comprising a first opening 103 located on one side (the top side) thereof and a second opening 105 located on an opposite side (the bottom side) thereof. The heat-transfer unit 11 is mounted in the first opening 103 of the light-transmissive housing 10. The heater 3 is mounted to a bottom side of the heat-transfer unit 11. The light-emitting device 5 is mounted inside the light-transmissive housing 10. The light-emitting device 5 and the heater 3 are electrically coupled to a power supply unit (not shown). The heater 3 is controlled to heat the heat-transfer unit 11. The light-emitting device 5 is used to radiate a light source in all directions, for example, 360 degrees, that penetrates the light-transmissive housing 10 to generate a light decoration or a lighting effect.

Referring to FIGS. 1-4, a user can place an aroma capsule 12 on the heat-transfer unit 11 of the heat-activated aroma diffuser 1. When switched on the power supply unit (not shown), the heater 3 generates heat that is then transferred by the heat-transfer unit 11 to heat the aroma capsule 12, causing the aroma capsule 12 to release an aromatic vapor. At the same time, the light-emitting device 5 emits light to create a lighting effect.

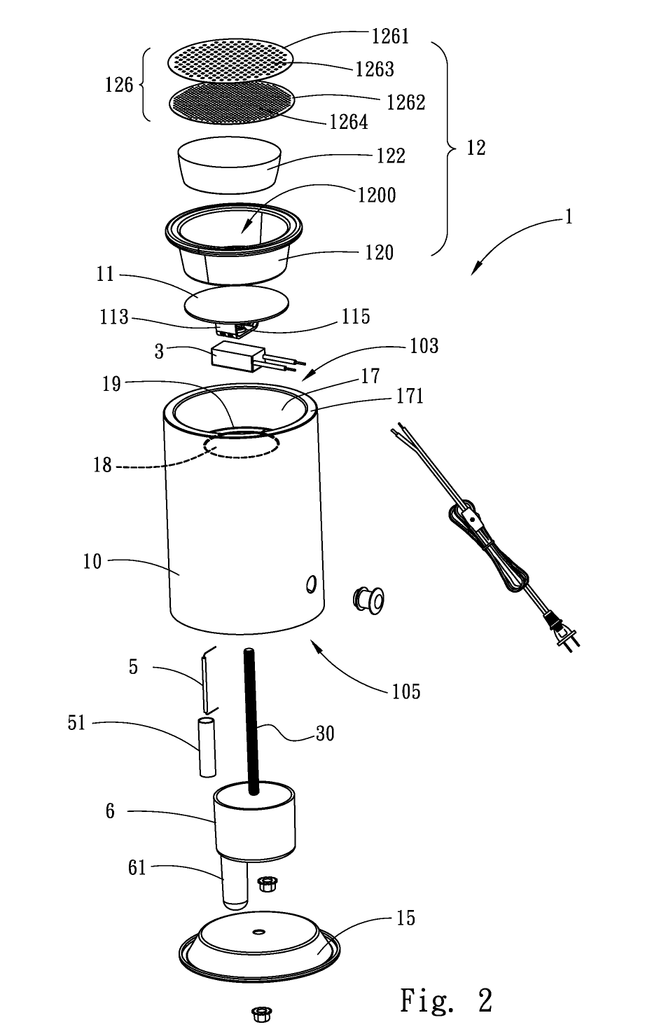

Referring to FIGS. 1 and 2, the light-emitting device 5 is a LED light-emitting device comprising a plurality of LED chips arranged in an array for radiating a light source in 360 degrees.

Referring to FIGS. 1 and 2, the light-emitting device 5 is surrounded by a protective tube 51. The protective tube 51 surrounds and protects the light-emitting device 5. The protective tube 51 is a light-transmitting or translucent material selected from a plastic material, such as PP plastic tube, or a light-transmissive material selected from glass, silicone and the like.

Referring to FIGS. 2-4, the heat-activated aroma diffuser 1 further comprises a light guide 6. The light guide 6 or the light-transmissive housing 10 is selected from a transparent or translucent material, such as plastics, porous ceramics, glass, silicone, or the light-transmissive housing 10 can be configured to provide a plurality of holes. In the present preferred embodiment, the light guide 6 is made of polypropylene (PP). The light guide 6 is mounted inside the light-transmissive housing 10. The light-emitting device 5 is mounted in the light guide 6. In the present preferred embodiment, the light-emitting device 5 can be directly connected to an external power source, or coupled to an internal power source. When the light-emitting device 5 is activated to emit light, the emitted light is transmitted through the light guide 6 to create a halo light soft decorative effect. At the same time, the light-transmissive housing 10 emits an aromatic vapor out of the heat-activated aroma diffuser 1 to create a good atmosphere. Thus, the heat-activated aroma diffuser 1 works as a night lamp, reminding the user that the heat-activated aroma diffuser 1 is in use. The light guide 6 in the present preferred embodiment is selected from polypropylene (PP).

Referring to FIGS. 1 and 2, the light guide 6 further comprises an extension hood 61 extended from one side of the light guide 6. In this embodiment, the extension hood 61 is a tubular member downwardly extended from the bottom side of the light guide 6 to surround the light-emitting device 5, or to surround the protective tube 51 that has the light-emitting device 5 mounted therein. Thus, the 360-degrees light emitted by the light-emitting device is guided by the extension hood 61 toward the light-transmissive housing 10 to produce beautiful soft lighting.

Further, the heat-transfer unit 11 is disposed at the bottom side of the aroma capsule 12 without surrounding the periphery of the aroma capsule 12 so that the user's hand can access to the aroma capsule 12 conveniently.

Referring to FIGS. 1 and 2, the heat-transfer unit 11 comprises a bracket 113 located at a bottom side thereof. The bracket 113 comprises a mounting hole 115. The heat-activated aroma diffuser 1 further comprises a connection unit 30 and a base 15. The base 15 is fastened to the second opening 105 of the light-transmissive housing 10. The heater 3 has one side thereof attached to the bottom side of the heat-transfer unit 11. The heater 3 is mounted in the bracket 113 at the bottom side of the heat-transfer unit 11. The connection unit 30 has one end thereof fastened to the base 15, and an opposite end thereof fastened to the mounting hole 115 of the bracket 113 and stopped against an opposite side of the heater 3. In this embodiment, the mounting hole 115 is a screw hole; the connection unit 30 is a screw rod with its one end threaded into the mounting hole 115 and its opposite end fastened to the base 15 by a screw nut.

Thus, the heat-transfer unit 11 is firmly secured inside the light-transmissive housing 10; the heater 3 and the heat-transfer unit 11 are held in positive contact with each other for heat transfer.

Referring to FIGS. 1-4, the light-transmissive housing 10 further comprises a concave taper 17, an inner flange 19, and a through hole 18. The concave taper 17 slopes downwardly inwards from the topmost edge 171 of the light-transmissive housing 10. The inner flange 19 of the light-transmissive housing 10 is horizontally located on the lowest center of the concave taper 17. The through hole 18 is defined in the center of the concave taper 17 and surrounded by the inner flange 19. The through hole 18 accommodates the bracket 113 of the heat-transfer unit 11, allowing the heat-transfer unit 11 to be supported on the inner flange 19 of the light-transmissive housing 10 over the through hole 18. Thus, the concave taper 17, the inner flange 19 and the heat-transfer unit 11 define a trough for accommodating an aroma capsule 12. The design of the concave taper 17 facilitates loading or removal of an aroma capsule 12.

In this embodiment, the heat-transfer unit 11 is a circular metal plate for transferring heat evenly to the aroma capsule 12.

In this embodiment, the light-transmissive housing 10, the concave taper 17, the inner flange 19 and the heat-transfer unit 11 can be made of light transmissive ceramic, plastic, glass or porous metal in integrity in the shape of a hopper for accommodating an aroma capsule 12. Further, the design of the concave taper 17 facilitates loading or removal of an aroma capsule 12.

The heat-transfer unit 11 is made of a heat conducting material, such as metal, ceramic or heat conductive glass for efficiently transferring heat energy from the heater 3 to the heat-transfer unit 11.

Referring to FIGS. 1-4, the heat-activated aroma diffuser 1 is used with an aroma capsule 12. The aroma capsule 12 is placed on the heat-transfer unit 11. The heater 3 is connected to a power source and activated to generate heat. The generated heat energy is then transferred by the heat-transfer unit 11 to heat the aroma capsule 12, causing the aroma capsule 12 to release an aromatic vapor.

Referring to FIGS. 1-4, in the preferred embodiment of the present invention, the aroma capsule 12 comprises a heat-transfer container 120 defining a top opening 1200, an aromatic substance 122 held in the heat-transfer container 120, and a breathing film bonded to the heat-transfer container 120 over the top opening 1200 and the aromatic substance 122 to allow moisture vapor to be transmitted through the material.

Referring to FIGS. 1-2, in one embodiment of the present invention, the breathing film 126 comprises a fiber fixation layer 1261 and a microporous layer 1262 of excellent moisture permeability. The fiber fixation layer 1261 defines therein multiple first pores 1263. The microporous layer 1262 defines therein multiple second pores 1264. The fiber fixation layer 1261 and the microporous layer 1262 are bonded together to form the breathing film 126 with the breathable and waterproof function. The microporous layer 1262 is preferably made from thermoplastic elastomer (TPE) compound. The fiber fixation layer 1261 is preferably made from polyester. The polyester fiber fixation layer 1261 and the thermoplastic elastomer (TPE) microporous layer 1262 are bonded together to form the breathing film 126 that is breathable. The breathable aperture of the first pores 1263 is greater than the breathable aperture of the second pores 1264, facilitating outward transmittance of air. The breathing film 126 is bonded to the heat-transfer container 120 over the top opening 1200. Through the second pores 1264 of the thermoplastic elastomer (TPE) microporous layer 1262, the aroma capsule 12 provides waterproof and moisture transmissive functions, keeping the aromatic substance 122 fresh.

The thermoplastic elastomer (TPE) microporous layer 1262 and the fiber fixation layer 1261 can be bonded together using hot-press fusion or high-frequency sealing technology. Since the first pores 1263 of the fiber fixation layer 1261 are relatively lager than the second pores 1264 of the thermoplastic elastomer (TPE) microporous layer 1262, the heated aromatic substance vapor molecules can go through second pores 1264 of the thermoplastic elastomer (TPE) microporous layer 1262 toward the first pores 1263 of the fiber fixation layer 1261.

In application, the aroma capsule 12 is heated in the heat-activated aroma diffuser 1 to release a pleasant smell at a temperature below 90° C. During the heating process, the fiber fixation layer 1261 does not shrink or expand the size and can keep the thermoplastic elastomer (TPE) microporous layer 1262 in shape, and thus, the surface of the breathing film 126 can be constantly maintained smooth without wrinkles. During the heating process, aromatic substance 122 keeps releasing aromatic vapor molecules that flow smoothly through the second pores 1264 of the thermoplastic elastomer (TPE) microporous layer 1262 and the first pores 1263 of the fiber fixation layer 1261 toward the atmosphere outside the aroma capsule 12, however, the liquid phase aromatic substance is prohibited from passing through the second pores 1264, ensuring safety application of the aroma capsule 12.

Further, the aromatic substance 122 is selected from the group consisting of aromatic wax, perfume, balsam, sesame oil mixture and essential oil. In the case an aromatic wax is used for the aromatic substance 122 of the aroma capsule 12, the heat-activated aroma diffuser 1 simply needs to heat the aromatic wax to the melting point of the aromatic wax, causing the aromatic wax to release aromatic vapor molecules through the breathing film 126 toward the outside open air. In the case a sesame oil mixture or essential oil is used for the aromatic substance 122 of the aroma capsule 12, the sesame oil mixture or essential oil is heated to release aromatic vapor molecules through the breathing film 126 toward the outside open air. When compared to related existing commercial products, the breathing film 126 of the present invention will not deform during the heating process, and the released aromatic vapor molecules can flow through the breathing film 126 toward the outside open air efficiently.

Further, the heat-transfer container 120 is made from polymers. In one embodiment, the heat-transfer container 120 is made from plastics selected from the group consisting of polyester plastic, poly chloro terephthalate glycol (PCTG), polyethylene terephthalate (PET), propylthiouracil (PTU) and polypropylene (PP). The plastic heat-transfer container 120 does not melt or deform at the above-mentioned heating temperature, and can efficiently transfer heat energy from the heat-activated aroma diffuser 1 to the aromatic substance 122. In one embodiment of the present invention, the heat-transfer container 120 is made from PCTG, capable of transferring heat energy from the heat-activated aroma diffuser 1 to the aromatic substance 122 for causing the aromatic substance 122 to release a pleasant smell. Further, the heat-transfer container 120 has excellent ductility and toughness and is not easy to rapture. It will not be damaged when heating by the heat-activated aroma diffuser, and can efficiently transfer heat energy from the heat-activated aroma diffuser to the aromatic substance. When compared to fragile pottery and glass heat-transfer containers of conventional heat-activated aroma diffusers, the heat-transfer container 120 has the advantage that the top opening 1200 of the heat-transfer container 120 can easily be bonded with the breathing film 126, i.e., the thermoplastic microporous layer 1262 of the breathing film 126 can be bonded to the top opening 1200 of the heat-transfer container 120 by heat or with an adhesive. The bonding effect is better than the prior art design. After bonding, the breathing film 126 will not fall off.

Referring to FIGS. 1-4, in application, the user simply needs to put the aroma capsule 12 in the heat-activated aroma diffuser 1, and then to electrically conduct the heat-activated aroma diffuser 1, melting the aromatic substance 122 (such as aromatic wax or essential oil) to emit aromatic vapor molecules. In the heating process, the emitted aromatic vapor molecules go from the aroma capsule 12 through the second pores 1264 of the microporous layer 1262 of the breathing film 1261 and then the first pores 1263 of the fiber fixation layer 1261 to the surrounding air while the breathing film 1261 stays in shape and keeps breathing.

After the aromatic substance 122 is used up, the heat-transfer container 120 can be thrown away or recycled, and a new aroma capsule 12 can be put in the table-top heat-activated aroma diffuser 1. Thus, the user's hand will not be contaminated by the aromatic substance 122. Further, the user does not necessary to clean the table-top heat-activated aroma diffuser 1. Even if the table-top heat-activated aroma diffuser 1 falls accidentally during application, the melted aromatic substance 122 of the aroma capsule 12 is still held in the heat-transfer container 120 by the breathing film 126 and will not flow to the outside of the table-top heat-activated aroma diffuser 1 to cause contamination or dangers.

Although a particular embodiment of the present invention has been described in detail for purposes of illustration, various modifications and enhancements may be made without departing from the spirit and scope of the invention. Accordingly, the invention is not to be limited except as by the appended claims.

What the invention claimed is:

1. A table-top heat-activated aroma diffuser, comprising:
light-transmissive housing comprising a first opening located on one side thereof and a second opening located on an opposite side thereof;
a heat-transfer unit mounted in said first opening of said light-transmissive housing;
a heater mounted to a bottom side of said heat-transfer unit; and
a light-emitting device mounted inside said light-transmissive housing.

2. The table-top heat-activated aroma diffuser as claimed in claim 1, wherein said light-emitting device is a LED light-emitting device.

3. The table-top heat-activated aroma diffuser as claimed in claim 1, further comprising a protective tube surrounding said light-emitting device, said protective tube being selectively made of light-transmissive or translucent material.

4. The table-top heat-activated aroma diffuser as claimed in claim 2, further comprising a protective tube surrounding said light-emitting device, said protective tube being selectively made of light-transmissive or translucent material.

5. The table-top heat-activated aroma diffuser as claimed in claim 3, further comprising a light guide selectively made of a light transmissive or translucent material and mounted inside said light-transmissive housing; said light-emitting device is mounted in said light guide.

6. The table-top heat-activated aroma diffuser as claimed in claim 4, further comprising a light guide selectively made of a light transmissive or translucent material and mounted inside said light-transmissive housing; said light-emitting device is mounted in said light guide.

7. The table-top heat-activated aroma diffuser as claimed in claim 5, further comprising an extension hood extended from one side of said light guide to surround said light-emitting device and said protective tube.

8. The table-top heat-activated aroma diffuser as claimed in claim 6, further comprising an extension hood extended from one side of said light guide to surround said light-emitting device and said protective tube.

9. The table-top heat-activated aroma diffuser as claimed in claim 1, wherein said heat-transfer unit comprises a bracket located at a bottom side thereof, said bracket comprising a mounting hole; heater has one side thereof attached to a bottom side of said heat-transfer unit; said heat-activated aroma diffuser further comprises a connection unit and a base, said base being fastened to said second opening of said light-transmissive housing, said connection unit having one end thereof fastened to said base and an opposite end thereof fastened to said mounting hole of said bracket and stopped against an opposite side of said heater.

10. The table-top heat-activated aroma diffuser as claimed in any of claim 1, further comprising an aroma capsule placed on said heat-transfer unit inside said light-transmissive housing, said aroma capsule comprising a heat-transfer container defining a top opening, an aromatic substance put in said heat-transfer container and a breathing film having a breathable function and bonded to said heat-transfer container over said top opening to hold said aromatic substance in said heat-transfer container, said breathing film comprising a fiber fixation layer defining therein a plurality of first pores and a microporous layer bonded to said fiber fixation layer in a stack and defining therein a plurality of second pores.

11. The table-top heat-activated aroma diffuser as claimed in claim 10, wherein said microporous layer is made from thermoplastic elastomer with said second pores defined therein; said fiber fixation layer is made out of polyester with said first pores defined therein.

12. The table-top heat-activated aroma diffuser as claimed in claim 11, wherein said fiber fixation layer is preferably made out of polyester, said polyester being selected from the group consisting of Tetoron and polyethylene terephthalate (PET); said thermoplastic elastomer is thermoplastic polyurethane (TPU); said heat-transfer container is selected from the material group consisting of plastic, polyester plastic, poly chloro terephthalate glycol (PCTG), polyethylene terephthalate (PET), propylthiouracil (PTU) and polypropylene (PP); said thermoplastic polyurethane (TPU) of said microporous layer of said breathing film is bonded to said heat-transfer container around said top opening.

13. The table-top heat-activated aroma diffuser as claimed in claim 10, wherein the breathable aperture of said first pores is greater than the breathable aperture of said second pores.
